(19) United States
(12) Patent Application Publication
(10) Pub. No.: US 2013/0184224 A1
(43) Pub. Date: Jul. 18, 2013

(54) METHODS FOR INHIBITING TUMOR GROWTH

(71) Applicant: National Taiwan University, Taipei City (TW)

(72) Inventors: Show-Li Chen, Taipei (TW); Hsuan-Hao Chen, Taipei (TW); Kuan-Liang Lin, Taipei (TW); Hsin-Hsiung Chen, Taipei (TW); Szu-Wei Chang, Taipei (TW)

(73) Assignee: NATIONAL TAIWAN UNIVERSITY, Taipei City (TW)

(21) Appl. No.: 13/736,466

(22) Filed: Jan. 8, 2013

Related U.S. Application Data
(63) Continuation-in-part of application No. 12/839,754, filed on Jul. 20, 2010.

(51) Int. Cl. A61K 38/17

(52) U.S. Cl. CPC A61K 38/1709
USPC 514/19.5; 514/19.2; 514/44 R

(57) ABSTRACT

The present invention provides a method for inhibiting androgen receptor (AR)-containing tumor cell growth in a subject in need thereof, comprising administrating to said subject a pharmaceutically effective amount of a damaged-DNA binding protein 2 (DDB2) and a pharmaceutically acceptable carrier. The present invention also provides a method for inhibiting androgen receptor (AR)-containing tumor cell growth in a subject in need thereof, comprising administrating to said subject a pharmaceutically effective amount of an expression vector comprising a gene encoding a damaged-DNA binding protein 2 (DDB2) and a pharmaceutically acceptable carrier. In a preferred embodiment, the expression vector is a plasmid vector.

### CROSS-REFERENCE TO RELATED APPLICATIONS

This application is a Continuation-in-part of the pending U.S. patent application Ser. No. 12/839,754 filed on Jul. 20, 2010, for which priority is claimed and is incorporated herein by reference in its entirety.

Although incorporated by reference in its entirety, no arguments or disclaimers made in the parent application apply to this divisional application. Any disclaimer that may have occurred during the prosecution of the above-referenced application(s) is hereby expressly rescinded. Consequently, the Patent Office is asked to review the new set of claims in view of the prior art of record and any search that the Office deems appropriate.

### FIELD OF THE INVENTION

The present invention relates to a method for inhibiting androgen receptor (AR)-containing tumor cell growth by using of damaged-DNA binding protein 2 (DDB2).

### BACKGROUND OF THE INVENTION

Recently, the male prostate cancer incidence increased year after year. According to the report of the Department of Health (DOH) of Taiwan, prostate cancer ranked seventh in both top ten fatal cancers and male top ten fatal cancers and cannot be ignored since it's minacity to males. Prostate cancer is formed due to malign hypertrophy of prostate cells, which might relate to genetic factors, lack of male hormones, diet, environmental factors and viral or bacterial infections.

The early symptom of prostate cancer is similar to which of benign prostatic hypertrophy (BPH). In early phase, patients feel difficultly to urinate or micturate frequently. When the urinary tract or the bladder neck gets invaded or obstructed, the symptoms like hematuria, incontinence, intermittent or weak urine flow, pain and burning sensation during urinating or ejaculation and urinary tract infection may appear. Also, prostate cancer often transfers to the bone and then causes bone pain, fractures or symptoms of spinal nerve compression. Prostate cancer can be divided into four phases in accordance with the degree of the distribution cancer cells:
Phase I: Tumor is located in the prostate, and the clinical examination is difficult to find cancer cells.
Phase II: Clinical examination detects prostate cancer in the lump, but the cancer is confined within the prostate capsule.
Phase III: Violation of prostate cancer cells outside the organization, such as: seminal vesicle, peripheral fat, urethra muscles and bladder neck.
Phase IV: Cancer metastasis, violations of the pelvis, lymph nodes or distant organs.

Diagnosis of prostate cancer usually means a “rectal examination”, that is by rectal examination by a physician to feel whether the prostate lumps or nodules phenomenon. Recently, because of advances in diagnostic techniques, the test of blood prostate specific antigen (PSA) and prostatic acid phosphatase (PAP) is widely used in routine medical examinations. This greatly enhances the efficiency of early diagnosis of prostate cancer and thus increases the cure rate. Inevitably, however, there was still a part of the patients who had prostate cancer and were detected late or get recurrence or metastasis after surgical resection.

The clinical treatments of prostate cancer are as follows:
1. Radical surgical treatment: For patients with earlier phase, resection of the prostate gland can eradicate all cancer cells within the prostate; but it is useless for those whose cancer cells metastasize and violate the lymph nodes. The operative mortality is low, but most patients lost their sexuality and suffered from urinary incontinence after the surgical operation.
2. Cryotherapy: Cryotherapy kills cancer cells by freezing them. It is a less aggressive therapy and patients need not to have general anesthesia. This therapy has the better effect than Radical surgical treatment when used in the treatment of cancer recurrence after radical treatment and also reduces postoperative urinary problems; however, it leads to increase the likelihood of sexual dysfunction to 90%.
3. Radiation therapy: This method uses radiation to destroy cancer cells and inhibit their growth and division by the treatment course of about 30 to 40 times of exposure and was usually applied to patients who can't perform the surgery. Radiation therapy is a local treatment that can only kill cancer cells in the treatment region, and the course of treatment will cause side effects and discomfort. There is new radiation treatment method which uses computer-knife radiosurgery for the treatment of localized tumors, and high doses of radiation can be accurately applied to the tumor to minimize side effects and reduce the damage to normal organ function. Course of treatment is without real-knife surgery, and the effect of surgical treatment can be achieved in about 3 to 5 times of operations. However, it is only for the treatment of local and costs much time for a single treatment. Moreover, it costs so much money.
4. Hormone therapy: Hormone therapy is applied to patients who are not suitable for surgical operation because of the metastasis of tumor. Most prostate tumor cells are very sensitive to androgen, and blockade of androgen-tumor cell interaction leads to the inhibition of tumor proliferation and metastasis. Clinically, the most common way of hormone therapy is testes removal or luteinizing hormone-releasing hormone analogues (LHRH analogues) treatment. However, hormone therapy is apt to cause flushing, decreased sexual desire, sexual dysfunction, osteoporosis and other side effects. Also, this therapy is only effective in treatment of one type of prostate cancer (androgen-dependent prostate cancer, ADPC) but not another (androgen-independent prostate cancer, AIPC).

Damaged-DNA binding protein 2 (DDB2, also named p48), a member of DDB1 and Cul4-associated factors (DCAFs), contains three WD40 domain and was originally found to involve in nucleotide excision repair along with damaged-DNA binding protein 1 (DDB1) (Takao, M., M. Abramic, et al., A 127 kDa component of a UV-damaged DNA-binding complex, which is defective in some xeroderma pigmentosum group E patients, is homologous to a slime mold protein. Nucleic Acids Res 21(17): 4111-8 (1993)). DDB2-deficient mice not only were hypersensitive to UV-induced skin carcinogenesis but also developed a high rate of malignant tumor in internal organ which indicate DDB2 function as a tumor suppressor (Itoh, T., S. Iwashita, et al., Ddb2 is a haploinsufficient tumor suppressor and controls spontaneous germ cell apoptosis. Hum Mol Genet 16(13): 1578-86 (2007)). In addition to DNA repair, DDB2 may function as a transcription factor to regulate gene expression. It had reported that DDB2 acts as a co-factor of E2F1 (Hayes, S., P. Shiyanov, et al., DDB, a putative DNA repair protein, can function as a transcriptional partner of E2F1. Mol Cell Biol 18(1):240-9 (1998)) and that associated with chromatin-acetylating transcription co-activator STAGA complex (SPT3-TAFII31-GCN5L acetylase complex). In contrast that DDB2 is considered as a tumor suppressor, recent study has reported that DDB2 is a candidate for oncogene in breast cancer which may contribute to breast tumor progression (Kattan, Z., S. Marchal, et al., Damaged DNA binding protein 2 play a role in breast cancer growth.” PLoS One 3(4): e2002 (2008)). However, the relationship between DDB2 and prostate cancer was not disclosed.

Ubiquitin E3 ligase contains two important functions; one for catalysis of isopeptide bond formation and the other for the recruitment of substrates to this catalytic activity. The cullin (CUL) family is evolutionarily conserved proteins that assemble a large family of cullin-dependent E3 ligase. The human cullin family includes CUL1, CUL2, CUL3, CUL4A, CUL4B, CUL5 and CULT. All cullins contain a conserved carboxy-terminal domain which binds to small RING finger protein: ROC1 (Regulator of Cullins-1, also called Rbx1) or ROC2 (Regulator of Cullins-2) (Petroski, M. D. and Deshaies, R. J., Function and regulation of cullin-RING ubiquitin ligases. Nat Rev Mol Cell Biol 6 (1), 9 (2005)). The small RING finger protein can recruit E2 ubiquitin-conjugating enzyme to proceed to ubiquitination. Cullin-dependent E3 ligase require to interact with an adaptor protein to target specific substrate, rather than binding to substrate directly as other E3 ligase. For example, CUL1-dependent ligase rely on interaction with an adaptor protein SKP1 (S-phase kinase-associated protein 1) to bridge an F-box protein to target specific substrate (Petroski, M. D. and Deshaies, R. J., Function and regulation of cullin-RING ubiquitin ligases. Nat Rev Mol Cell Biol 6 (1), 9 (2005)). CUL4 fuctions as ubquitin E3 ligase by recruiting ring finger protein (ROC1) and various substrate receptors. To target specific substrate, CUL4 utilizes the C-terminus to bind with ROC1 and the N-terminus to interact with linker protein (DDB1) which recruits various substrate receptors to target specific substrate. A well-known model is DDB2-DDB1-CUL4 complex which involves in NER (nucleotide excision repair) pathway after UV-irradiation. The DDB2-DDB1-CUL4 E3 ligase complex is recruited to the DNA lesion foci at the damaged DNA, and then ubiquitinates histone H2A, and H3, H4 (Wang, H. et al., Histone H3 and H4 ubiquitylation by the CUL4-DDB-ROC1 ubiquitin ligase facilitates cellular response to DNA damage. Mol Cell 22 (3), 383 (2006)) etc. After ubiquitination, the histones may dissolve from the damaged nucleosome that makes the damaged DNA exposed. Later, the NER pathway factor XPC (Xeroderma pigmentosum group C-complementing protein) is recruited to the damaged site and the NER pathway proceeds (Sugasawa, K. et al., UV-induced ubiquitylation of XPC protein mediated by UV-DDB-ubiquitin ligase complex. Cell 121 (3), 387 (2005)).

The turnover of androgen receptor (AR) plays an important role in AR protein regulation. There are three pathways reportedly to be involved in AR degradation. Firstly AR can be phosphrylated by PI3K/AKT and subsequently undergoes ubiquitination by MDM2 E3 ligase. After ubiquitination, AR was degraded through 26S proteasome (Gaughan, L. et al., Tip60 and histone deacetylase 1 regulate androgen receptor activity through changes to the acetylation status of the receptor. J Biol Chem 277 (29), 25904 (2002)). Secondly, androgen-induced AR translocation can be interfered by phosphatidylinositol-3,4,5-trisphosphate 3-phosphatase (PTEN). The interaction between AR and PTEN may expose the active site of the AR for the recognition of caspase-3, leading to AR degradation (Lin, H. K., Y. C. Hu, et al., Regulation of androgen receptor signaling by PTEN (phosphatase and tensin homolog deleted on chromosome 10) tumor suppressor through distinct mechanisms in prostate cancer cells. Mol Endocrinol 18(10): 2409-23. (2004)). Thirdly, in DDB1-CUL4B complex, AhR (dioxin receptor) can be activated in the presence of ligand (3-methylcholanthrene) and then interacts with aryl hydrocarbon receptor nuclear translocator (Arnt) to form heterodimer and translocate into nucleus. The heterodimer can associate with DDB1-CUL4B complex to assemble a functional E3 ligase. Sex steroid hormone receptor AR or ER can be a target substrate and ubiqutinated by this this E3 ligase complex (Ohtake, F. et al., Dioxin receptor is a ligand-dependent E3 ubiquitin ligase. Nature 446 (7135), 562 (2007)). However, the relationship between DDB2 and AR was not disclosed.

### SUMMARY OF THE INVENTION

The present invention is directed to a method for inhibiting androgen receptor (AR)-containing tumor cell growth in a subject in need thereof, comprising administrating to said subject a pharmaceutically effective amount of a damaged-DNA binding protein 2 (DDB2) and a pharmaceutically acceptable carrier.

The present invention also is directed to a method for inhibiting androgen receptor (AR)-containing tumor cell growth in a subject in need thereof, comprising administrating to said subject a pharmaceutically effective amount of an expression vector comprising a gene encoding a damaged-DNA binding protein 2 (DDB2) and a pharmaceutically acceptable carrier. In a preferred embodiment, the gene is SEQ ID NO: 5. In one embodiment, the expression vector is a plasmid vector.

### BRIEF DESCRIPTION OF THE DRAWINGS

FIGS. 1A-1C show that DDB2 interacts with androgen receptor (AR) reciprocally in vitro and in vivo.

FIGS. 1A and 1B show that DDB2 can directly interact with AR extracellularly.

FIG. 1C shows that DDB2 can also interact with AR in vivo.

FIGS. 2A-2D show that DDB2 induces the ubiquitin level of AR and DDB1-CUL4A is involved in AR protein stability.

FIG. 2A shows that DDB2 can ubiquitinize AR.

FIG. 2B shows that the increase of DDB1 lowers the expression of intracellular AR.

FIG. 2C shows that after leveling down the expression of DDB1 by shDDB1, the AR level measures higher than control.

FIG. 2D shows that after leveling down the expression of CUL4A by shCUL4A, the AR level measures higher than control. The quantitative data of AR protein is analyzed by UVP and presented as mean±SD.

FIG. 3 shows biological significance of DDB2 effects. LNCap and PC-3 cell are plated in 6-well dishes in complete medium, and cell numbers were counted per two days. Surviving cell numbers are counted by trypan blue staining. Means are shown for three experiments. Nuclear and cytoplasmic fractions are prepared from LNCap and PC-3 cells then analyzed by western blot.

FIGS. 4A-4B show that overexpression of DDB2 decreases AR protein level (FIG. 4A) but not that of AR mRNA (FIG. 4B). LNCaP cells are transfected with the pcDNA3.1-myc-DDB2 expression plasmid or control vector under the following conditions: electroporation for 24 h in culture medium containing charcoal-dextran stripped FBS (CDS), and then treatment with ethanol or 10 nM DHT for another 16 h. Total protein and RNA are isolated and analyzed by western blotting (FIG. 4A) and qPCR (FIG. 4B), respectively.

FIGS. 5A-5B show that knockdown of DDB2 gene expression increases AR protein level (FIG. 5A) but not RNA (FIG. 5B). LNCaP cells are transiently transfected with 200 pmol of oligonucleotides for silencing DDB2 (Applied Biosystems) or with control siScramble RNA and harvested 48 h later. Total protein and RNA are collected and analyzed by western blotting and qPCR.

FIGS. 6A-6B show that DDB2 causes the ubiquitination of AR.

FIG. 6A shows overexpression of DDB2 enhances the level of polyubiquitinated AR proteins. The expression vectors including Myc-DDB2 and FLAG-Ub are cotransfected into LNCaP cells. 24 h posttransfection, cells are cultured in the 10% FBS with 10 μM of MG132 or DMSO. The cell lysates are extracted and immunoprecipitated with antibodies against AR. The eluted protein complex is separated by SDS-PAGE and analyzed by western blotting with the antibodies indicated. The levels of AR and exogenous DDB2 are shown in the lower panel, and the polyubiquitinated forms of AR are shown in the upper panel.

FIG. 6B shows that DDB2 increases AR polyubiquitination both in EtOH and DHT. LNCaP cells are treated as described in panel A and cultured in EtOH or 10 nM DHT for 24 h and in the presence of MG132. The lower panel shows the protein levels in the whole-cell extract; upper panel represents polyubiquitinated AR.

FIGS. 7A-7B show that DDB2 inhibits AR-containing cancer cell growth.

FIG. 7A shows the effect of DDB2 on AR protein stability. LNCaP cells are electroporated with pcDNA3.1-myc-DDB2 expression plasmid or control vector for 24 h in 5% CDS medium, treated with 200 μg/mL cycloheximide (CHX) in the presence of 1 nM DHT, and then harvested for a western blot assay at different time as indicated. Band densities are quantified using a UVP

BioSpectrum-AC Imaging System.

FIG. 7B shows that deprived endogenous DDB2 increases AR protein stability both in EtOH (left) and DHT treatment (right). The AR protein level is normalized using GAPDH and in each control treatment is considered to be 100%, so that changes in density are measured as a percentage of each control treatment over time (lower panels).

FIGS. 8A-8B show that degradation of AR induces the apoptosis of cancer cell.

FIG. 8A shows that AR depletion in LNCaP cells induces apoptosis. Cells are transfected with shRNA targeted AR or Myc-DDB2, and then analyzed by flow cytometry 48 h posttransfection. Lower: AR and DDB2 protein levle; upper: cell cycle distribution, and the number of sub-G1 (%).

FIG. 8B shows that LNCaP cells are transfected with the expression plasmid of DDB2 or mock, respectively. After 48 h transfection, cells are collected and co-stained with Propidium Iodide (PI) and Annexin V. Flow cytometry analysis is performed and the graph shows the percentage of early (Annexin V positive/PI negative) plus late (Annexin V positive/PI positive) apoptotic cells.

### DETAILED DESCRIPTION OF THE INVENTION

As used herein in the specification, “a” or “an” may mean one or more. As used herein in the claim(s), when used in conjunction with the word “comprising”, the words “a” or “an” may mean one or more than one. The term “DDB2” used herein refers to damaged-DNA binding protein 2, “AR” refers to androgen receptor, “DDB1” refers to damaged-DNA binding protein 1, “CUL4A” refers to cullin 4A protein, “LNCaP” refers to androgen-dependent prostate tumor cell line which contains AR, and “PC-3” refers to androgen-independent prostate tumor cell line which is without AR. The term “GST” used herein refers to glutathione S-transferase, “FLAG” refers to FLAG® epitope “DYKDDDDK” (SEQ ID NO: 1), “Myc” refers to c-Myc epitope tag “EQKLISEEDL” (SEQ ID NO: 2), and “HA” refers to hemagglutinin epitope tag “YPYDVPDYA” (SEQ ID NO: 3). The term “siRNA” used herein refers to small interfering RNA, “shRNA” refers to small hairpin RNA, and “MG132” refers to proteasome inhibitor.

The present invention provides a method for treating a subject suffering from growth of androgen receptor (AR)-containing tumor cells, comprising administrating the subject an effective amount of damaged-DNA binding protein 2 (DDB2).

The protein DDB2 mentioned herein is one subunit of damaged-DNA binding protein complex. It can associate with DDB1 and CUL4A and forming DDB2-DDB1-CUL4A complex.

In the present invention, the DDB2 can interact with androgen receptor. By the participation of DDB1-CUL4A complex, the DDB2-DDB1-CUL4A protein complex ubiquatinates AR and let it degrade, and therefore, the growth of AR-dependent tumor cell is suppressed.

In the present invention, the preferable tumor cell whose growth is suppressed by an effective amount of DDB2 is prostate tumor cell. Prostate tumor cells are divided into two groups according to the presence of AR. In a preferred embodiment of the invention, the prostate tumor cell lines are LNCaP and PC-3; the former is the androgen-dependent prostate tumor cell line while the latter is androgen-independent. In summary, the method of the invention can suppress the growth of LNCaP androgen-dependent (AR-containing) prostate tumor cell line effectively.

In the present invention, the administration can be applied by any known methods. For example: it can be manufactured into liquid injection form and applied to organisms. In addition, the effective amount of DDB2 can also be delivered via oral route. Through the digestive system and circulatory system, it will be delivered to target locations.

The present invention also provides a cancer-diagnosing kit, comprising (a) DDB2 and (b) DDB2-biomarker complex. In the cancer-diagnosing kit of the present invention, the biomarker complex is chosen from the group including radioactive isotopes, fluorescent molecules, luminescent markers, enzymes and affinity molecules. The cancer-diagnosing kit can also contain a signal-detecting reagent chosen from the group including antibodies, enzymes, affinity molecules and chemical coloring elements. In the present invention, the preferable cancer-diagnosing kit is prostate cancer-diagnosing kit.

The present invention provides a method for inhibiting androgen receptor (AR)-containing tumor cells growth in a subject in need thereof, comprising administrating to said subject a pharmaceutically effective amount of composition comprising a damaged-DNA binding protein 2 (DDB2) and a pharmaceutically acceptable carrier. In one embodiment, DDB2 interacts with androgen receptor (AR). In another embodiment, the AR-containing tumor cell growth is inhibited by degradation of AR. In a preferred embodiment, the degradation of AR is induced by DDB2 via an ubiquitin mechanism. In one embodiment, the ubiquitin mechanism involves participation of DDB1-CUL4A (cullin4A) complex.

In one embodiment, the subject is an animal. Preferably, the subject is a mammal More preferably, the subject is a human. In one embodiment, the tumor cells are prostate tumor cells. In a preferred embodiment, the method of the present invention further treats prostate cancer.

The present invention may be used to treat, alleviate, ameliorate, relieve, delay onset of, inhibit progression of, reduce severity of, and/or reduce incidence of one or more symptoms or features of a disease, disorder, and/or condition induced by the AR-containing tumor cell or the prostate cancer. In some embodiments, the present invention can be used to treat cancer and/or cancer cells. The “cancer cells” or “tumor cell”, as used herein, can be in the form of a tumor, exist alone within a subject, or be cell lines derived from a cancer. As used herein, the term “cell” includes either the singular or the plural of the term. As used herein, the term “inhibit cell growth” means the slowing or ceasing of cell division and/or cell expansion. This term also includes the inhibition of cell development or increases in cell death.

As used herein, “DDB2” refers to a protein or a gene encoding the protein. In one embodiment, the protein sequence of DDB2 is SEQ ID NO: 4. In another embodiment, the gene encoding DDB2 is SEQ ID NO: 5.

A “pharmaceutically effective amount” is an amount effective to prevent, lower, stop or reverse the development of, or to partially or totally alleviate the existing symptoms of a particular condition for which the subject being treated. Determination of a therapeutically effective amount is well within the capability of those skilled in the art.

The composition comprising the DDB2 can be administered to the subject by many routes and in many regimens that will be well known to those in the art. In some embodiments, the DDB2 is administered intravenously, intramuscularly, subcutaneously, topically, orally, or by inhalation. Through the digestive system and circulatory system, it will be delivered to target locations.

The composition comprising the DDB2 may be formulated for administration via sterile aqueous solution or dispersion, aqueous suspension, oil emulsion, water in oil emulsion, site-specific emulsion, long-residence emulsion, sticky-emulsion, microemulsion, nanoemulsion, liposomes, microparticles, microspheres, nanospheres, nanoparticles, minipumps, and with various natural or synthetic polymers that allow for sustained release. The compounds comprising the DDB2 may also be formulated into aerosols, tablets, pills, sterile powders, suppositories, lotions, creams, ointments, pastes, gels, hydrogels, sustained-delivery devices, or other formulations used in drug delivery.

The pharmaceutically acceptable carriers are determined in part by the particular composition being administrated, as well as by particular method used to administer the composition. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a subject. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

The present invention also provides a method for inhibiting androgen receptor (AR)-containing tumor cell growth in a subject in need thereof, comprising administrating to said subject a pharmaceutically effective amount of an expression vector comprising a nucleic acid sequence encoding a damaged-DNA binding protein 2 (DDB2) and a pharmaceutically acceptable carrier. In one embodiment, the expression vector is a plasmid vector. In a preferred embodiment, the gene is SEQ ID NO: 5. In one embodiment, DDB2 interacts with androgen receptor (AR). In another embodiment, the AR-containing tumor cell growth is inhibited by degradation of AR. In a preferred embodiment, the degradation of AR is induced by DDB2 via an ubiquitin mechanism. In one embodiment, the ubiquitin mechanism involves participation of DDB1-CUL4A (cullin4A) complex.

In one embodiment, the subject is an animal. Preferably, the subject is a mammal. More preferably, the subject is a human. In one embodiment, the tumor cells are prostate tumor cells. In a preferred embodiment, the method of the present invention further treats prostate cancer.

In some embodiment, the present invention can be applied to gene therapy. The expression vector of the present invention can comprise a gene encoding the DDB2, the gene can be administered in vivo to promote expression of its encoded protein, by constructing it as part of an appropriate nucleic acid expression vector and administering it so that it becomes intracellular, e.g., by use of a retroviral vector, or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus, etc. Alternatively, a nucleic acid can be introduced intracellularly and incorporated within host cell DNA for expression, by homologous recombination.

The term “expression vector”, as used here, is meant to include any type of genetic vector containing a polynucleotide sequence coding for a DDB2 gene product in which part or all of the DDB2 nucleic acid is capable of being transcribed and subsequently translated into a protein.

As referred to herein, the term “encoding” is intended to mean that the gene or nucleic acid may be transcribed in a cell, e.g., when the nucleic acid is linked to appropriate control sequences such as a promoter in a suitable vector (e.g., an expression vector) and the vector is introduced into a cell. Such control sequences are well known to those skilled in the art.

As used herein, the term “gene” means a nucleic acid which encodes a protein or functional fragment thereof. The term “nucleic acid” is intended to mean natural and synthetic linear and sequential arrays of nucleotides and nucleosides, e.g., in cDNA, genomic DNA (gDNA), mRNA, and RNA, oligonucleotides, oligonucleosides and derivatives thereof. It will also be appreciated that such nucleic acids can be incorporated into other nucleic acid chains referred to as “vectors” by recombinant-DNA techniques such as cleavage and ligation procedures.

### EXAMPLES

The examples below are non-limiting and are merely representative of various aspects and features of the present invention.

### Example 1

### Protein-Protein Interaction Between DDB2 and AR In Vitro and In Vivo

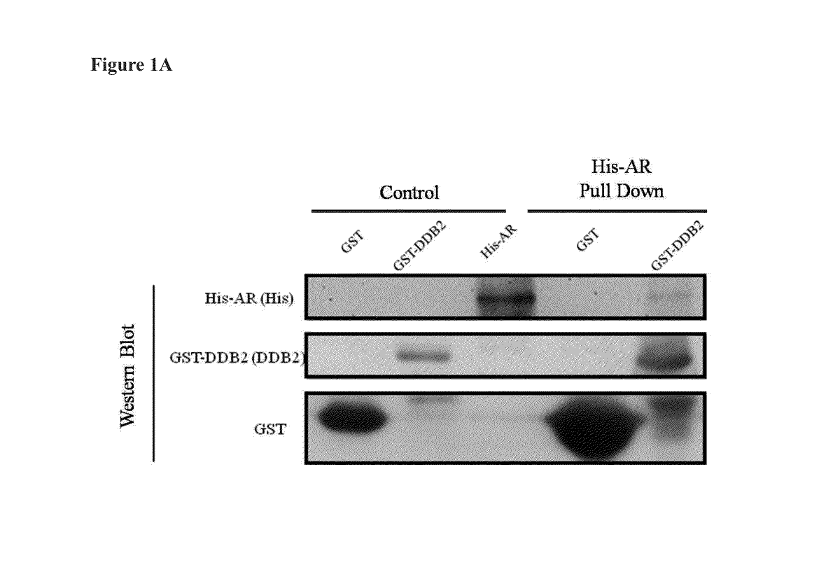

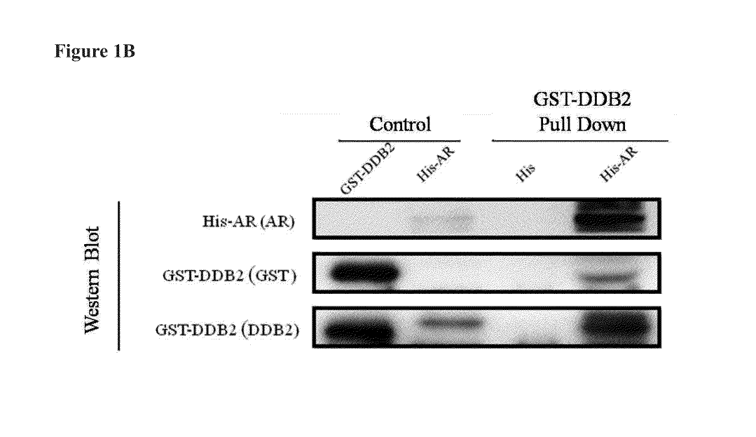

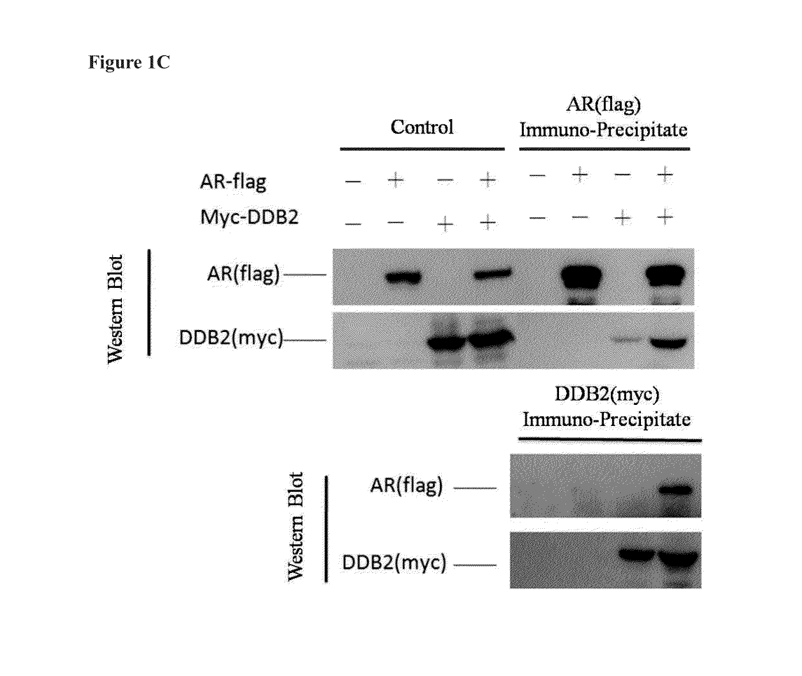

To test whether DDB2 could directly interact with AR reciprocally, His-AR fusion protein synthesized by E. coli was purified using Ni-NTA beads and incubated with purified GST-DDB2 fusion protein in GST pull down assay. The co-purified complex was separated with SDS-PAGE and western-blotting was performed with anti-His for AR or anti-GST for DDB2 protein, respectively (FIG. 1A). Also, the recombinant GST-DDB2 protein was purified and then incubated with purified His-AR protein. The eluted proteins were analyzed by western-blotting (FIG. 1B). The results showed that the AR-bound beads could be pulled down with the purified His-AR protein (FIG. 1A); and vice versa (FIG. 1B). These results indicated that AR directly interacts with DDB2 in vitro.

Furthermore, to test whether DDB2 associated with AR reciprocally in vivo, the Myc-tagged DDB2 gene and FLAG-tagged AR plasmids were co-transfected into NEK 293T cells by the standard calcium phosphate method. After 48h, the cell lysate was collected and immuoprecipitated with anti-FLAG to AR or anti-Myc to DDB2 protein, respectively. And then, the co-immunoprecipitated complexes were analyzed with the antibodies indicated. The Western blotting analysis data showed that there was an interaction signal compared with the control group. The same result was also observed by reciprocal experiment (FIG. 1C). These results indicated that DDB2 can interact with AR in vivo. Taken together, DDB2 could interact reciprocally with AR in vitro and in vivo, implying that DDB2 was a novel AR-binding protein.

### Example 2

### Effect of DDB2 on AR Protein Ubiquitination

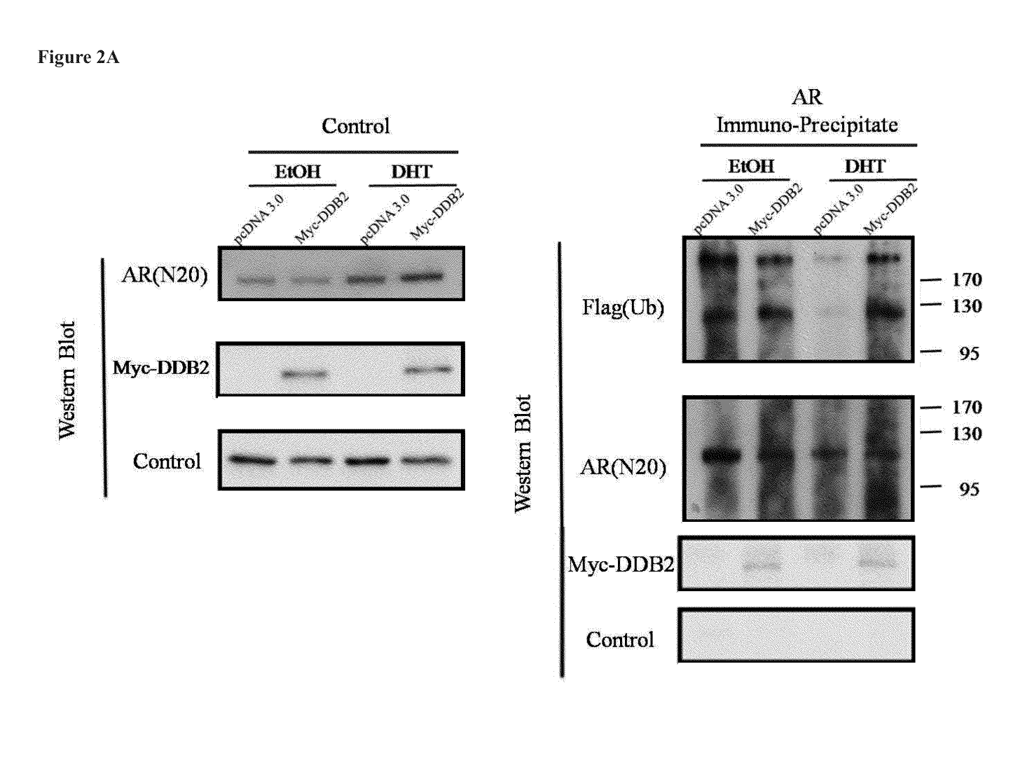

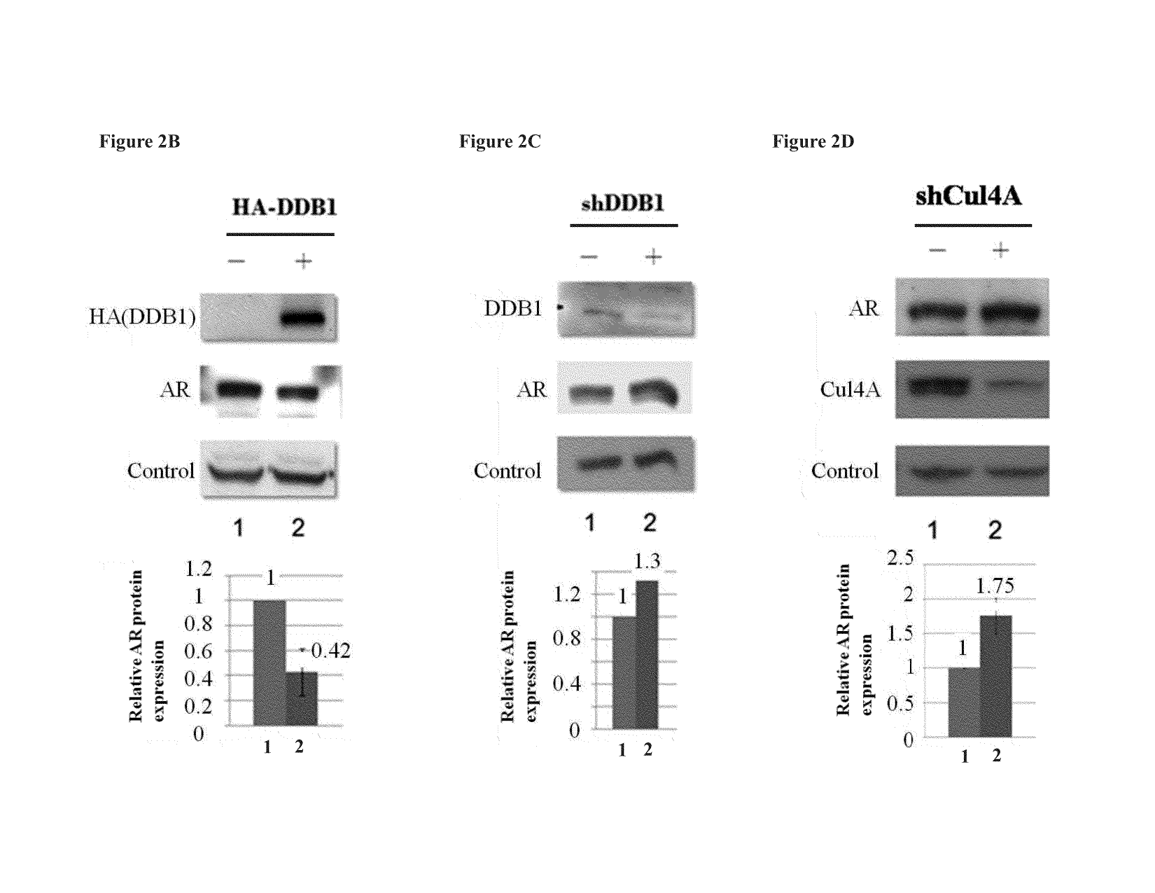

To further investigate the effect of DDB2 on ubiquitylation level of AR, equilmolar amounts of AR (10 μg), Myc-tagged DDB2 (10 μg) and FLAG-tagged ubiquitin (5 μg) were transfected into NEK 293T cells. After 48 hours, the cells were treated with 20 μM MG132 for 6 hours incubation. The cell lysate was used to proceed with immunoprecipitation with anti-AR antibody and then subjected to Western blotting analysis with anti-FLAG antibody to examine the ubiquitylation level of AR protein. The result showed that DDB2 can induce AR ubiquitin level compared with vector control (FIG. 2A).

Recently, it had been reported that DDB1-Cul4 E3 ligase was involved in the degradation of AR protein and DDB2 was found to be a member of DCAFs which could interact with DDB1 and functions as a substrate receptor of DDB1-Cul4 E3 ligase complex. In this regard, DDB1 was expressed in prostate cancer cell line, LNCaP to test whether DDB2-degrading AR involved in DDB1-CUL4 degradation pathway. The result showed that DDB 1 could reduce AR protein level (FIG. 2B) Moreover, siRNA was used to knockdown endogenous DDB1 (FIG. 2C) or Cul4A (FIG. 2D), and AR was increased in siDDB1 or siCUL4A expressing cells in response. These results suggested that DDB1-Cul4A pathway might be involved in AR degradation.

### Example 3

### Overexpression of DDB2 Results in Reducing Growth Rate of Androgen-Dependent Prostate Cancer Cells

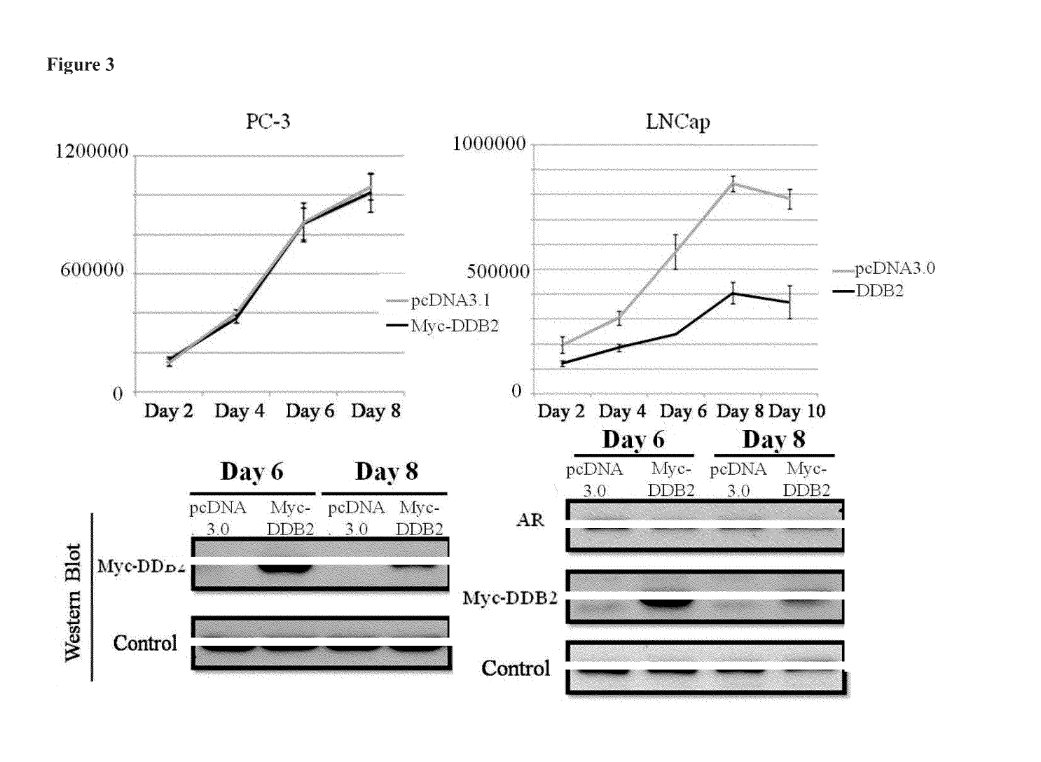

To elucidate the effect of overexpression of DDB2 on the growth rate of AR-dependent prostate cancer cell, the present invention introduced DDB2 gene (SEQ ID NO: 5) into AR-containing prostate cancer cell lines. Two kinds of prostate cancer cells including LNCaP and PC-3 cells, which were androgen-dependent and androgen-independent respectively, were seeded into 24-well plates and transfected with Myc-DDB2, or pcDNA3.0 as a control by electroporation. After plating and maintaining the cells in the medium containing FBS, total cell numbers were counted every 2 days using a hemocytometer and trypan blue exclusion. The cell lysated at day 6 and day 8 were also collected and Western blotting was performed to detect the protein levels as indicated. The data shown in FIG. 3 demonstrate that DDB2 could degrade AR resulting in the decreased growth rate in AR-containing cells (LNCaP) but not in AR-null cells (PC-3), implying that DDB2 was involved in AR protein degradation.

### Example 4

### DDB2 Triggers the Degradation of AR Protein

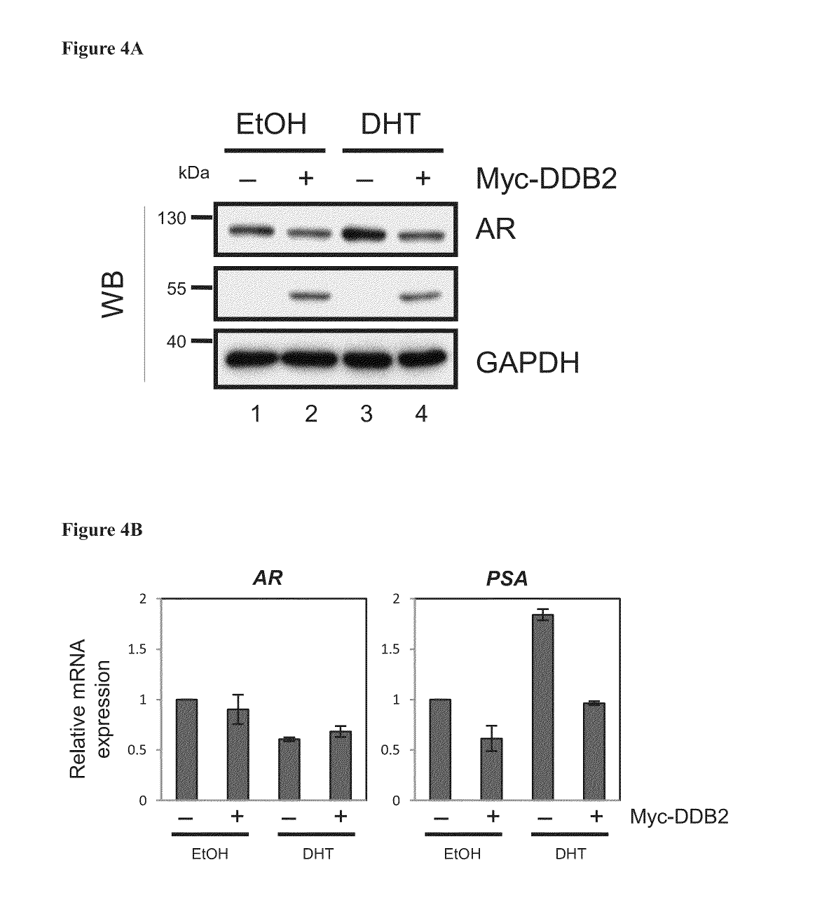

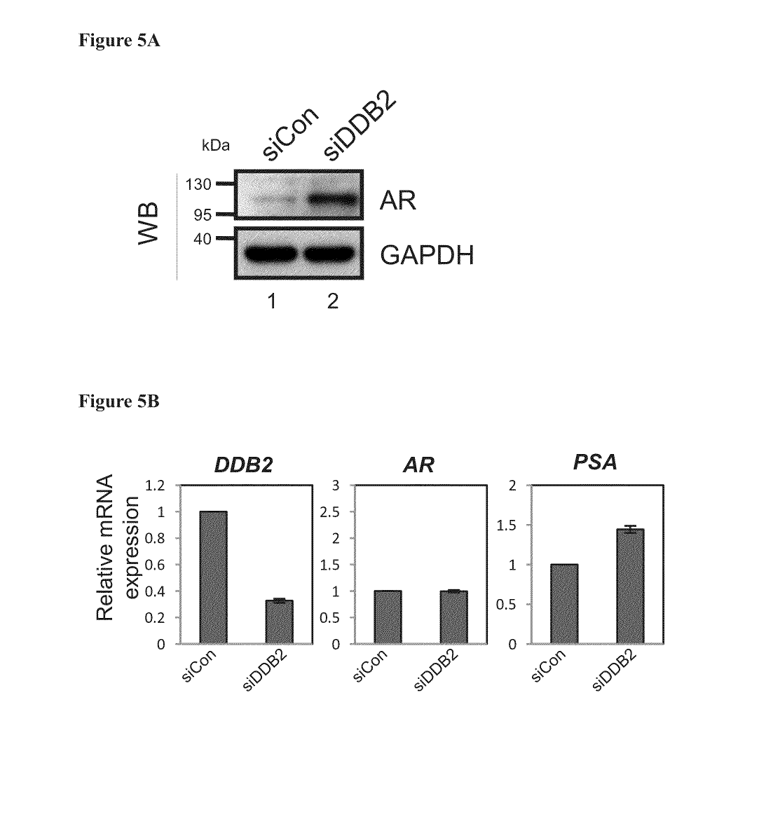

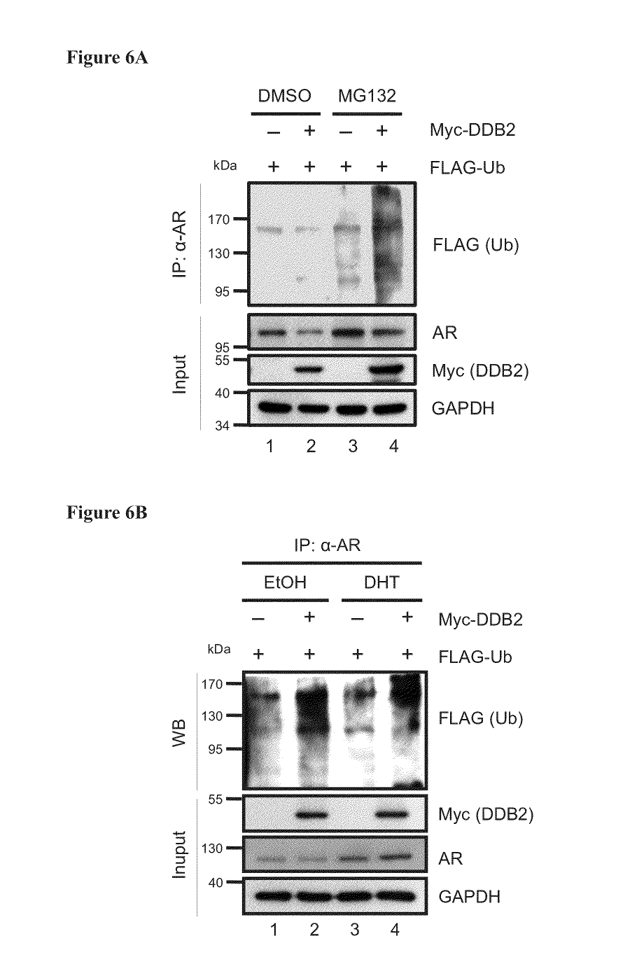

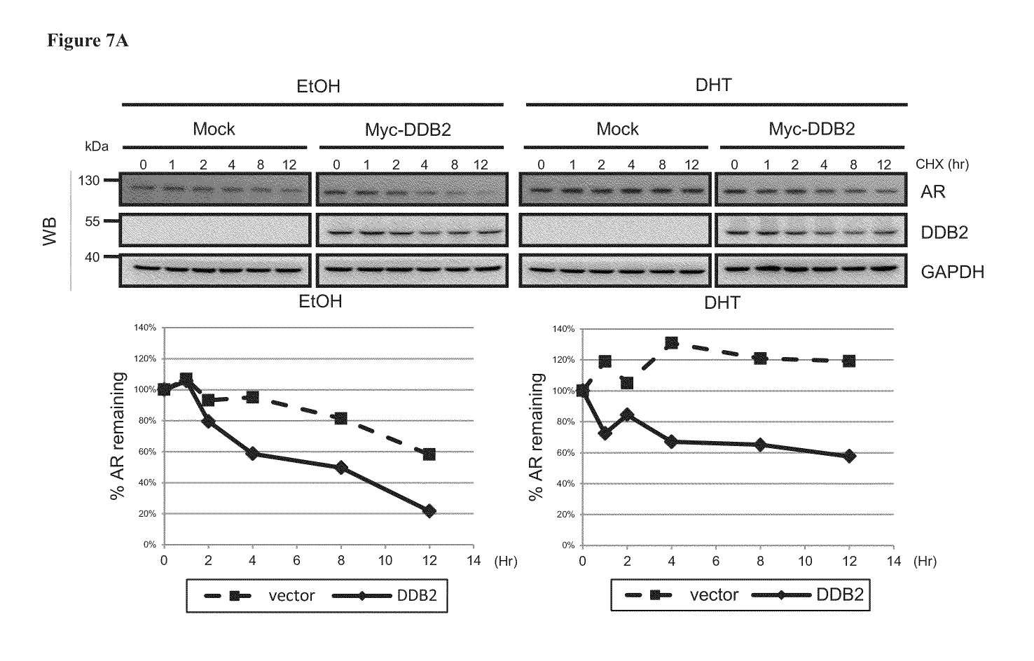

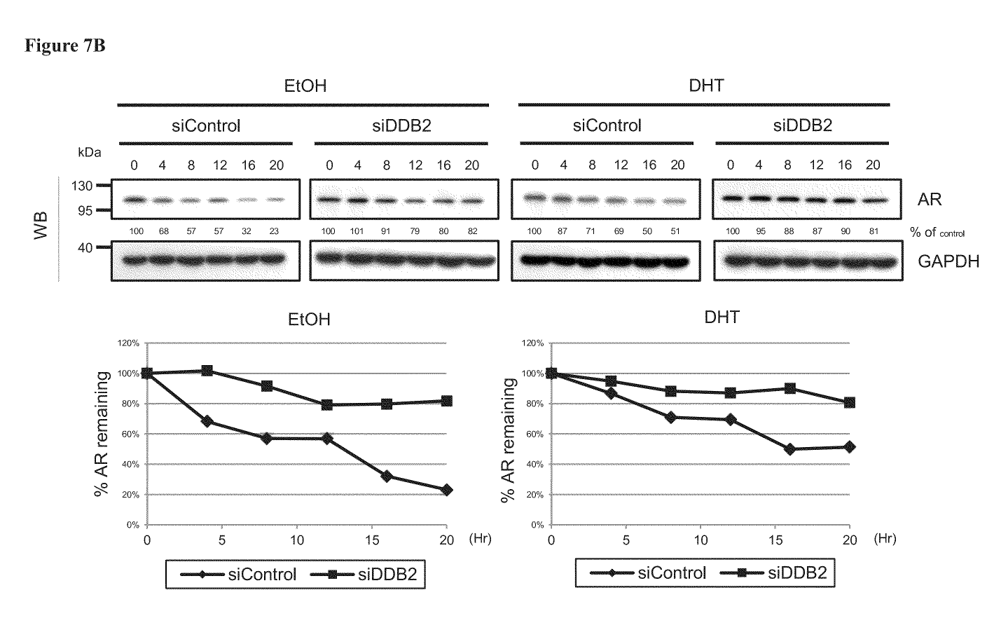

The DDB1-CUL4 E3 ligase complex containing DDB2 had ubiquitinating function. The present invention further investigated whether DDB2 was involved in AR degradation. As shown in FIG. 4A, overexpression of DDB2 decreased AR protein level both in EtOH and DHT (FIG. 4A, lanes 2 and 4) and coupled with the decrease PSA mRNA expression, but not affected AR mRNA level (FIG. 4B). The results implied that DDB2 regulated the AR in a translational level. Conversely, AR protein level coupled with its targeted gene-PSA mRNA expression was increased in LNCaP cells transfected with siRNA against DDB2 to knockdown endogenous DDB2 expression (FIGS. 5A and 5B). To further examine whether DDB2-degrading AR was proteasome-dependent, the present invention measured the ubiquitination of AR in the presence or absence of proteasome inhibitor-MG132. As shown in FIG. 6A, AR protein level was decreased in DDB2-containing cells without MG132 (compared lower panel lane 2 with lane 1), however the decreased AR protein was rescued in the presence of MG132 but AR was highly polyubiquitinated (upper, lane 4 to lane 2), indicating that DDB2-degrading AR was proteasome-dependent. To further investigate whether DHT may affect DDB2-induced AR polyubiquitination, the results showed that DDB2 caused polyubiquitination of AR both in the absence or presence of DHT (FIG. 6B, lane 2 and 4). To further assess whether DDB2 regulated the turnover rate of AR proteins, as shown in FIG. 7A, the half-life of AR protein was shorter in DDB2-treated LNCaP cells than in control cells both treated with DHT (right panel) or ethanol (EtOH; left panel). On the contrary, the half-life of AR protein was increased both in EtOH and DHT treatment when DDB2 was knockdowned by siRNA (FIG. 7B). Nevertheless AR protein remained more stable in cells treated with DHT than in cells not treated with DHT. This result was consistent with the result shown in FIG. 7A (compare lane 1 with lane 3). Taken together, these results suggested that DDB2 promoted AR degradation.

### Example 5

### DDB2 Decreases Cell Growth Rate in AR-Containing Prostate Cancer Cells

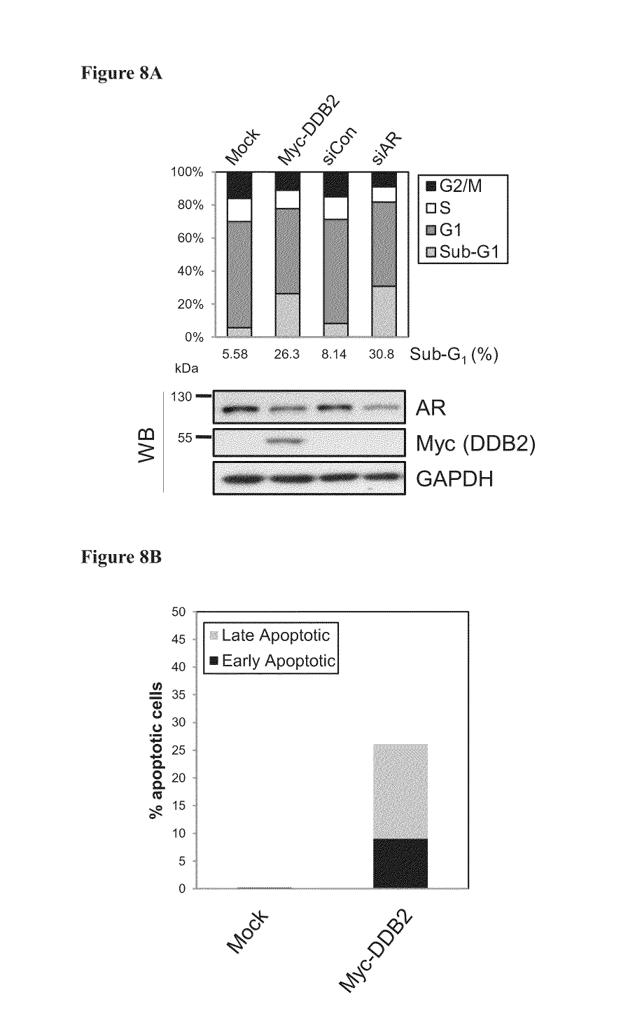

The central role of AR in the development and growth of androgen-dependent prostate cancers is highlighted by a study showing that knockdown of AR expression by shRNA reduced proliferation of LNCaP cells (Cheng H et al., Short hairpin RNA knockdown of the androgen receptor attenuates ligand-independent activation and delays tumor progression, Cancer Res. 2006, November 1; 66(21):10613-20). Based on above results, the growth rate of the androgen-dependent cell line LNCaP were significantly reduced by overexpression of DDB2 while PC-3 cells, which were androgen-independent, were not affected. The cell cycle analysis demonstrated that LNCaP cells treated shRNA targeted AR or transfected with DDB2 gene (SEQ ID NO: 5) presented 30.8% or 26.3% apoptosis, respectively (FIG. 8A). Apoptosis induced by DDB2 in LNCaP cells was also examined by flow cytometry using annexin V (FIG. 8B). Hence, AR could be the major factor to affect the growth rate of LNCaP cells. In conclusion, DDB2 could degrade AR protein and thereby decrease the growth rate of AR-expressing cells (LNCaP) but not that of AR-null cells (PC3). Thus, DDB2 was involved in AR protein degradation and consequently in cell growth inhibition.

One skilled in the art readily appreciates that the present invention is well adapted to carry out the objects and obtain the ends and advantages mentioned, as well as those inherent therein. The animals, processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method for inhibiting androgen receptor (AR)-containing tumor cell growth in a subject in need thereof, comprising administrating to said subject a pharmaceutically effective amount of a damaged-DNA binding protein 2 (DDB2) and a pharmaceutically acceptable carrier.

2. The method of claim 1, wherein the DDB2 interacts with androgen receptor (AR).

3. The method of claim 1, wherein the AR-containing tumor cell growth is inhibited by degradation of AR.

4. The method of claim 3, wherein the degradation of AR is induced by DDB2 via an ubiquitin mechanism.

5. The method of claim 4, wherein the ubiquitin mechanism involves participation of DDB1-CUL4A (cullin4A) complex.

6. The method of claim 1, wherein the subject is a mammal

7. The method of claim 1, wherein the subject is a human.

8. The method of claim 1, wherein the tumor cell is a prostate tumor cell.

9. The method of claim 1, which further treats prostate cancer.

10. A method for inhibiting androgen receptor (AR)-containing tumor cell growth in a subject in need thereof, comprising administrating to said subject a pharmaceutically effective amount of an expression vector comprising a gene encoding a damaged-DNA binding protein 2 (DDB2) and a pharmaceutically acceptable carrier.

11. The method of claim 10, wherein the expression vector is a plasmid vector.

12. The method of claim 10, wherein the gene is SEQ ID NO: 5.

13. The method of claim 10, wherein the DDB2 interacts with androgen receptor (AR).

14. The method of claim 10, wherein the AR-containing tumor cell growth is inhibited by degradation of AR.

15. The method of claim 14, wherein the degradation of AR is induced by DDB2 via an ubiquitin mechanism.

16. The method of claim 15, wherein the ubiquitin mechanism involves participation of DDB1-CUL4A (cullin4A) complex.

17. The method of claim 10, wherein the subject is a mammal.

18. The method of claim 10, wherein the subject is a human.

19. The method of claim 10, wherein the tumor cell is a prostate tumor cell.

20. The method of claim 10, which further treats prostate cancer.


SEQUENCE LISTING (5 sequences)

SEQ ID NO: 1 - protein, 8 aa, Artificial Sequence
  FLAG
DYKDDDDK

SEQ ID NO: 2 - protein, 10 aa, Artificial Sequence
  Myc
EQKLISEEDL

SEQ ID NO: 3 - protein, 9 aa, Artificial Sequence
  HA
YPYDVPDYA

SEQ ID NO: 4 - protein, 427 aa, Homo sapiens
MAPKKRPETQKTSEIVLRPRNKRSRSPLELEPEAKKLCAKGSGPSRRCDSDCLWVGLAGPQILPPCRSIVRTLHQHKLGRASWPSVQQGLQQSFLHTLDSYRILQKAAPFDRRATSLAWHPTHPSTVAVGSKGGDIMLWNFGIKDKPTFIKGIGAGGSITGLKFNPLNTNQFYASSMEGTTRLQDFKGNILRVFASSDTINIWFCSLDVSASSRMVVTGDNVGNVILLNMDGKELWNLRMHKKKVTHVALNPCCDWFLATASVDQTVKIWDLRQVRGKASFLYSLPHRHPVNAACFSPDGARLLTTDQKSEIRVYSASQWDCPLGLIPHPHRHFQHLTPIKAAWHPRYNLIVVGRYPDPNFKSCTPYELRTIDVFDGNSGKMMCQLYDPESSGISSLNEFNPMGDTLASAMGYHILIWSQEEARTRK

SEQ ID NO: 5 - DNA, 1284 bp, Homo sapiens
atggctcccaagaaacgcccagaaacccagaagacctccgagattgtattacgccccaggaacaagaggagcaggagtcccctggagctggagcccgaggccaagaagctctgtgcgaagggctccggtcctagcagaagatgtgactcagactgcctctgggtggggctggctggcccacagatcctgccaccatgccgcagcatcgtcaggaccctccaccagcataagctgggcagagcttcctggccatctgtccagcaggggctccagcagtcctttttgcacactctggattcttaccggatattacaaaaggctgccccctttgacaggagggctacatccttggcgtggcacccaactcaccccagcaccgtggctgtgggttccaaagggggagatatcatgctctggaattttggcatcaaggacaaacccaccttcatcaaagggattggagctggagggagcatcactgggctgaagtttaaccctctcaataccaaccagttttacgcctcctcaatggagggaacaactaggctgcaagactttaaaggcaacattctacgagtttttgccagctcagacaccatcaacatctggttttgtagcctggatgtgtctgctagtagccgaatggtggtcacaggagacaacgtggggaacgtgatcctgctgaacatggacggcaaagagctttggaatctcagaatgcacaaaaagaaagtgacgcatgtggccctgaacccatgctgtgattggttcctggccacagcctccgtagatcaaacagtgaaaatttgggacctgcgccaggttagagggaaagccagcttcctctactcgctgccgcacaggcatcctgtcaacgcagcttgtttcagtcccgatggagcccggctcctgaccacggaccagaagagcgagatccgagtttactctgcttcccagtgggactgccccctgggcctgatcccgcaccctcaccgtcacttccagcacctcacacccatcaaggcagcctggcatcctcgctacaacctcattgttgtgggccgatacccagatcctaatttcaaaagttgtaccccttatgaattgaggacgatcgacgtgttcgatggaaactcagggaagatgatgtgtcagctctatgacccagaatcttctggcatcagttcgcttaatgaattcaatcccatgggggacacgctggcctctgcaatgggttaccacattctcatctggagccaggaggaagccaggacacggaagtga